(19) United States
(12) Patent Application Publication
(10) Pub. No.: US 2026/0301922 A1
(43) Pub. Date: Oct. 1, 2026

(54) INFORMATION PROCESSING APPARATUS, METHOD, AND PROGRAM

(71) Applicant: FUJIFILM Corporation, Tokyo (JP)

(72) Inventors: Kazuma YOKOI, Kanagawa (JP); Shinichi KOJIMA, Kanagawa (JP)

(73) Assignee: FUJIFILM Corporation, Tokyo (JP)

(21) Appl. No.: 19/558,493

(22) Filed: Mar. 6, 2026

(30) Foreign Application Priority Data
Mar. 27, 2025 (JP) 2025-054303

(51) Int. Cl. G16H 30/20; A61B 6/03; G06T 7/00; G06T 7/90

(52) U.S. Cl. CPC G16H 30/20; G06T 7/0012; G06T 7/90; A61B 6/032; G06T 2207/10081; G06T 2207/30004

(57) ABSTRACT

The processor is configured to acquire a first image representing first information and a second image representing second information for the same subject, and derive an information image including the first information as a brightness component and including the second information as a color component.

### CROSS-REFERENCE TO RELATED APPLICATION

The present application claims priority from Japanese Patent Application No. 2025-054303, filed on Mar. 27, 2025, the entire disclosure of which is incorporated herein by reference.

### BACKGROUND

### Technical Field

The present disclosure relates to an information processing apparatus, a method, and a program.

### Related Art

As a technique for effectively using information from X-ray groups having a plurality of energy distributions, photon counting computed tomography (PCCT) including a photon counting type detector that employs a photon counting method and dual energy (DE) CT are known.

In the PCCT and the DECT, since the absorption characteristics of radiation are different for each substance, a substance decomposition image in which substances included in the subject are discriminated can be obtained. In addition, for example, a substance decomposition image for each of two base substances set in advance, such as water and iodine, is acquired, and weighting calculation processing is performed using the two substance decomposition images, so that various images such as a virtual monochromatic image (VMI), an electron density image, an effective atomic number image, and a specific substance image (iodine map) can be acquired.

In addition, a method has been proposed in which, by using a scatter diagram in which a concentration of each base substance used for the substance decomposition is set as an axis and in which pixels of a substance decomposition image output by the substance decomposition are plotted according to which concentration of the base substance they correspond to, an analysis image in which a brightness value or a color density is changed is generated, or visibility of the image is improved (see JP2020-039872A and WO2016/158234A).

Since the virtual monochromatic image is an image close to an existing CT image, information (hereinafter, referred to as density information) representing attenuation of the substance included in the virtual monochromatic image is easily visible. However, since information (Z information) on an atomic number, which is a feature of the substance, is not conspicuous in one image, it is necessary to acquire the virtual monochromatic images at a plurality of energy levels. In addition, the electron density image is also an image close to the existing CT image, but is a special-purpose image in which the Z information is erased. Further, the effective atomic number image and the specific substance image are images with strong statistical noise, and thus are difficult to handle.

### SUMMARY OF THE INVENTION

The present disclosure has been made in view of the above circumstances, and an object of the present disclosure is to enable visibility of types of a plurality of substances while maintaining attenuation (density) information in one image.

According to the present disclosure, an information processing apparatus comprises:
a processor, and
the processor is configured to:
acquire a first image representing first information and a second image representing second information for the same subject; and
derive an information image including the first information as a brightness component and including the second information as a color component.

In the information processing apparatus according to the present disclosure, the first image may be a virtual monochromatic image derived at an energy level at which contour lines of pixel values are parallel to a longitudinal direction of a distribution of signal values of a first base substance and a second base substance in a scatter diagram in which the first base substance and the second base substance serve as axes.

In the information processing apparatus according to the present disclosure, the second image may be a substance type image representing a type of a substance in the virtual monochromatic image, which is derived by using substance decomposition information derived based on a distribution of the signal values in a direction intersecting a direction in which the pixel values of the virtual monochromatic image change in the scatter diagram.

In the information processing apparatus according to the present disclosure, the intersecting direction may be an orthogonal direction.

In the information processing apparatus according to the present disclosure, the first information may be a brightness component of the virtual monochromatic image, and the second information may be information representing the type of the substance in the substance type image.

According to the present disclosure, an information processing method comprises:
via a computer,
acquiring a first image representing first information and a second image representing second information for the same subject; and
deriving an information image including the first information as a brightness component and including the second information as a color component.

According to the present disclosure, there is provided an information processing program causing a computer to execute:
a procedure of acquiring a first image representing first information and a second image representing second information for the same subject; and
a procedure of deriving an information image including the first information as a brightness component and including the second information as a color component.

The technology of the present disclosure may be applied to a program product.

According to the present disclosure, types of a plurality of substances can be visible while maintaining attenuation (density) information in one image.

### BRIEF DESCRIPTION OF THE DRAWINGS

FIG. 1 is a schematic configuration diagram of a medical image capturing system comprising an information processing apparatus according to an embodiment of the present disclosure.

FIG. 2 is a diagram showing a hardware configuration of the information processing apparatus according to the present embodiment.

FIG. 3 is a diagram showing a functional configuration of the information processing apparatus according to the present embodiment.

FIG. 4 is a diagram showing an example of a scatter diagram.

FIG. 5 is a diagram showing a functional configuration of an image derivation unit.

FIG. 6 is a diagram for describing contour lines of pixel values of a virtual monochromatic image.

FIG. 7 is a diagram showing a virtual monochromatic image.

FIG. 8 is a diagram showing a structure of a phantom.

FIG. 9 is a diagram for describing derivation of substance classification information.

FIG. 10 is a diagram for describing the derivation of a substance type index image.

FIG. 11 is a diagram for describing the derivation of a substance type index image.

FIG. 12 is a diagram showing the substance type index image.

FIG. 13 is a diagram showing a substance type index color image.

FIG. 14 is a diagram showing a brightness reflecting substance color image.

FIG. 15 is a diagram showing a chroma saturation-decreased brightness reflecting substance color image.

FIG. 16 is a flowchart showing processing performed in the present embodiment.

### DETAILED DESCRIPTION

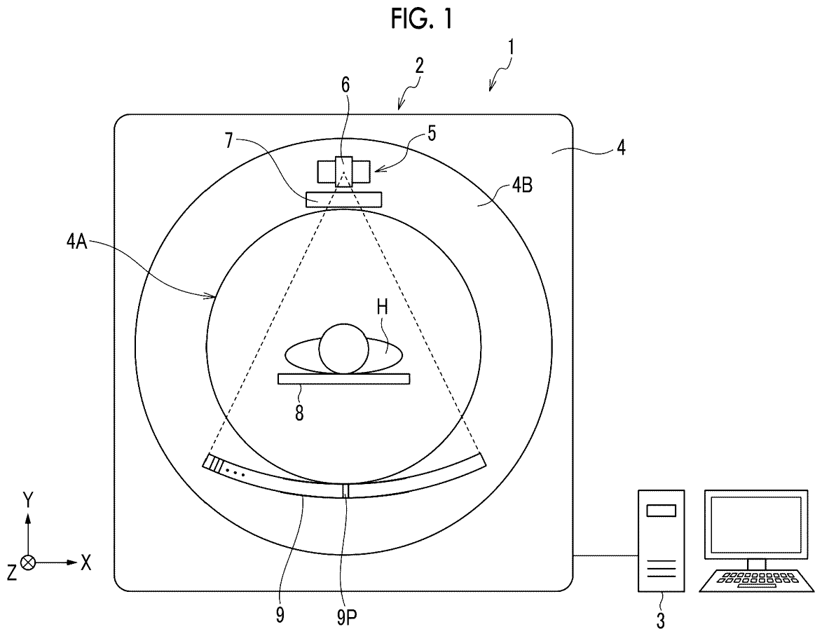

An embodiment of the present disclosure will be described in detail below with reference to the accompanying drawings. First, an example of a configuration of a medical image capturing system comprising an information processing apparatus of the present embodiment will be described. FIG. 1 is a schematic configuration diagram of the medical image capturing system comprising the information processing apparatus of the present embodiment.

A medical image capturing system 1 of the present embodiment comprises a CT apparatus 2 and a console 3, as shown in FIG. 1. The CT apparatus 2 comprises a gantry 4 and a patient table 8. In the following description, a horizontal direction in FIG. 1 is referred to as an X-axis, a vertical direction is referred to as a Y-axis, and a direction orthogonal to an XY plane is referred to as a Z-axis.

The gantry 4 has an opening portion 4A, and a subject H to be imaged is disposed within the opening portion 4A while being placed on the patient table 8. The gantry 4 and the patient table 8 are configured to move relative to each other in a Z-axis direction.

Inside the gantry 4, a radiation source 5 including a radiation tube 6 and a bowtie filter 7, and a detector 9 are disposed to face each other with the subject H interposed therebetween. The bowtie filter 7 optimizes an exposure dose by increasing the dose near a center and reducing the dose in the peripheral areas, in order to suppress the exposure dose in peripheral portions. Radiation emitted from the radiation tube 6 is shaped by the bowtie filter 7 into a beam shape suitable for a size of the subject H and is then emitted to the subject H. The detector 9 detects the radiation that has been transmitted through the subject H, and generates projection data corresponding to a photon count of the detected radiation. As one example, the detector 9 of the present embodiment is a photon-counting detector in which a plurality of detection elements 9P that detect photon energy, which is the energy of photons of incident radiation, are disposed in an arc shape centered on a focal point of the radiation tube 6. In the present embodiment, the detector 9 detects the photon energy of incident radiation by dividing the photon energy of incident radiation into a plurality of energy bins.

It should be noted that, in the present embodiment, X-rays are used as an example of the radiation, but the present disclosure is not limited to this, and γ-rays or the like may also be used.

The radiation source 5 and the detector 9 are rotated around the subject H by a rotation drive unit (not shown) of the gantry 4. As the radiation irradiation from the radiation source 5 and the detection of the radiation by the detector 9 are repeatedly performed in conjunction with the rotation of the radiation source 5 and the detector 9, data (hereinafter referred to as projection data) regarding the subject H is acquired for each radiation projection path. The projection data acquired by the detector 9 is output to the console 3 and stored in a storage of the console 3. A value of the data corresponding to each detection element 9P, which is a minimum unit of the projection data, is the count of photons detected by the detection element 9P. The projection data is acquired individually for each of the energy bins.

The dose of the radiation emitted from the radiation source 5, a rotation speed of the gantry 4, a relative movement speed between the gantry 4 and the patient table 8, and the like are set by the console 3 based on acquisition conditions for acquiring projection data, which are input by a user such as a technologist.

The console 3 of the present embodiment performs control related to acquisition of projection data, generation of medical images, control related to substance discrimination, and the like. The console 3 is an example of the information processing apparatus of the present disclosure.

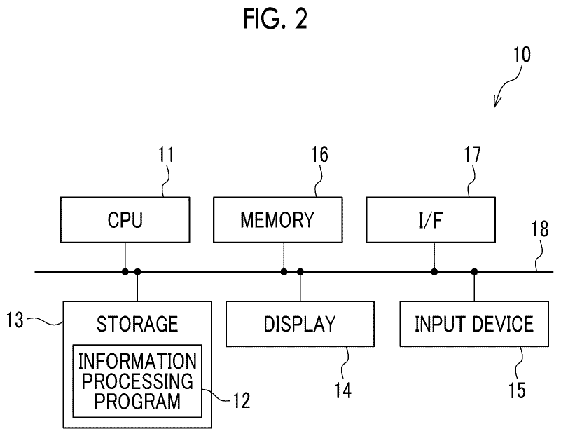

Next, the information processing apparatus according to the present embodiment will be described. First, a hardware configuration of the information processing apparatus according to the present embodiment will be described with reference to FIG. 2. As shown in FIG. 2, an information processing apparatus 10 is a computer, such as a workstation, a server computer, and a personal computer, and comprises a central processing unit (CPU) 11, a non-volatile storage 13, and a memory 16 as a temporary storage area.

In addition, the information processing apparatus 10 comprises a display 14, an input device 15, and an interface (I/F) 17. The CPU 11, the storage 13, the display 14, the input device 15, the memory 16, and the I/F 17 are connected to a bus 18. The CPU 11 is an example of a processor in the present disclosure.

The storage 13 is implemented using a hard disk drive (HDD), a solid-state drive (SSD), a flash memory, or the like. An information processing program 12 installed in the information processing apparatus 10 is stored in the storage 13 as a storage medium. The CPU 11 reads the information processing program 12 from the storage 13, loads the information processing program 12 into the memory 16, and executes the loaded information processing program 12.

The display 14 is a device that displays various screens, and is, for example, a liquid crystal display or an electro luminescence (EL) display.

The input device 15 is used by the user to input scan conditions for acquiring projection data, instructions related to generation, display, and the like of images, various kinds of information, and the like. Examples of the input device 15 include various switches, buttons, a touch panel, a touch pen, a keyboard, a mouse, and the like. The display 14 and the input device 15 may be integrated into a touch panel display.

The I/F 17 performs communication of various kinds of information with the rotation drive unit (not shown) of the gantry 4, the radiation source 5, and the detector 9 via wired communication or wireless communication.

The information processing program 12 is stored in a storage device of a server computer connected to a network or in a network storage in a state accessible from the outside and is downloaded to and installed in a computer that constitutes the information processing apparatus 10 in response to a request. Alternatively, the information processing program 12 is distributed by being recorded on a recording medium such as a digital versatile disc (DVD) or a compact disc read-only memory (CD-ROM), and is installed in a computer that constitutes the information processing apparatus 10 from the recording medium.

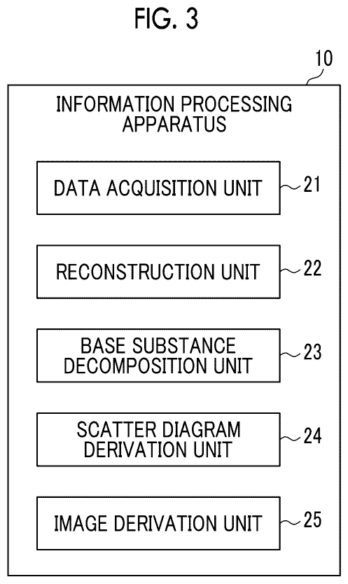

Next, a functional configuration of the information processing apparatus according to the present embodiment will be described. FIG. 3 is a diagram showing the functional configuration of the information processing apparatus according to the present embodiment. As shown in FIG. 3, the information processing apparatus 10 comprises a data acquisition unit 21, a reconstruction unit 22, a base substance decomposition unit 23, a scatter diagram derivation unit 24, and an image derivation unit 25. The CPU 11 executes the information processing program 12 to function as the data acquisition unit 21, the reconstruction unit 22, the base substance decomposition unit 23, the scatter diagram derivation unit 24, and the image derivation unit 25.

The data acquisition unit 21 acquires the projection data from the CT apparatus 2 via the I/F 17. For example, in a case in which four energy bins are set in the detector 9, the data acquisition unit 21 acquires the projection data for each of the four energy bins.

The reconstruction unit 22 derives a linear attenuation coefficient image for each energy bin by performing reconstruction processing on the projection data acquired by the data acquisition unit 21 for each energy bin.

The base substance decomposition unit 23 derives a base substance image by using the linear attenuation coefficient corresponding to the base substance and the linear attenuation coefficient image derived by the reconstruction unit 22. In the present embodiment, water and iodine are used as the base substances, and the base substance image for water and the base substance image for iodine are derived. Water is an example of the first base substance in the present disclosure, and iodine is an example of the second base substance in the present disclosure.

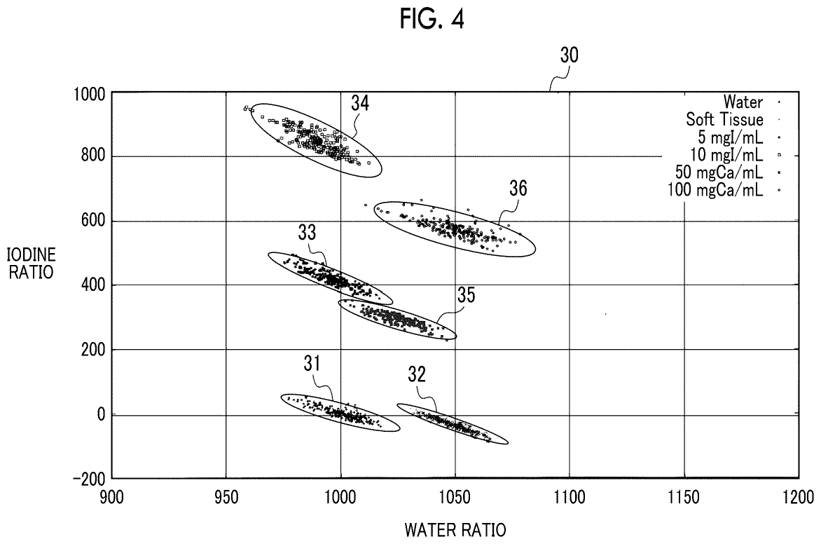

The scatter diagram derivation unit 24 derives a scatter diagram in which water as the first base substance and iodine as the second base substance are axes. Then, a signal value of the substance included in the linear attenuation coefficient image, which is represented by the concentrations of water and iodine, is plotted as the scatter diagram. FIG. 4 is a diagram showing an example of the scatter diagram. In a scatter diagram 30 shown in FIG. 4, the horizontal axis indicates a water ratio (base substance concentration) that is a presence ratio of water to iodine, and the vertical axis indicates an iodine ratio (base substance concentration) that is a presence ratio of iodine to water. The horizontal axis is a water ratio axis, and the vertical axis is an iodine ratio axis.

In the scatter diagram 30, the signal values represented by water and iodine for a plurality of substances are distributed in an elliptical shape. The scatter diagram derivation unit 24 approximates the distribution of the signal values for each substance by elliptical regions 31 to 36. The region 31 is a distribution of pixels of water, the region 32 is a distribution of pixels of soft tissue, the region 33 is a distribution of pixels of iodine of 5 mg/mL, the region 34 is a distribution of pixels of iodine of 10 mg/mL, the region 35 is a distribution of pixels of calcium of 50 mg/mL, and the region 36 is a distribution of pixels of calcium of 100 mg/mL.

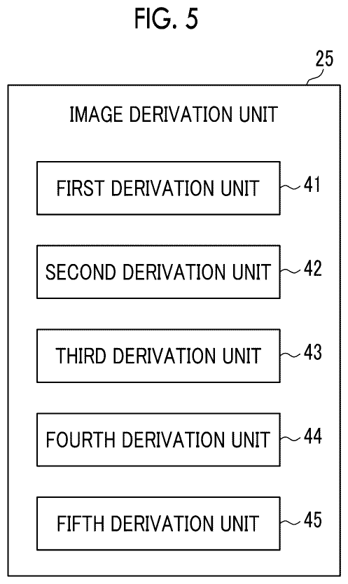

FIG. 5 is a diagram showing a functional configuration of the image derivation unit. As shown in FIG. 5, the image derivation unit 25 includes a first derivation unit 41, a second derivation unit 42, a third derivation unit 43, a fourth derivation unit 44, and a fifth derivation unit 45.

The first derivation unit 41 derives the virtual monochromatic image, which is a virtual linear attenuation image captured at a predetermined energy level, by weighted addition of the base substance image of water and the base substance image of iodine. In the present embodiment, the energy level of the virtual monochromatic image is set such that contour lines of pixel values in the virtual monochromatic image are parallel to a longitudinal direction of the elliptical regions 31 to 36 in the scatter diagram 30. The longitudinal direction of the elliptical region is a major axis direction of the elliptical region. As the energy level of the virtual monochromatic image, for example, 70 keV can be used as a tube voltage, but the present disclosure is not limited to this. The term “parallel” does not mean that the lines are completely parallel, and the lines may be substantially parallel to the longitudinal direction of the elliptical regions 31 to 36. The term “substantially parallel” indicates, for example, approximately ±5°.

An equation of the contour line for any pixel value (HU value) in the virtual monochromatic image is shown in Equation (1). In Equation (1), x and y are values of water and iodine after the substance decomposition, and μWi and μIo are linear attenuation coefficients of water and iodine, respectively. Equation (2) is obtained by modifying Equation (1) to represent the value y of iodine as a function f(x, HU) of the value x of water and the HU value.

$${μ}_{Wt} x + {μ}_{Io} y = \frac{{μ}_{Wt} ( HU + 1000 )}{1000} \qquad ( 1 )$$

$$y = f ( x , HU ) = \frac{\frac{{μ}_{Wt} ( HU + 1000 )}{1000} - {μ}_{Wt} x}{{μ}_{Io}} \qquad ( 2 )$$

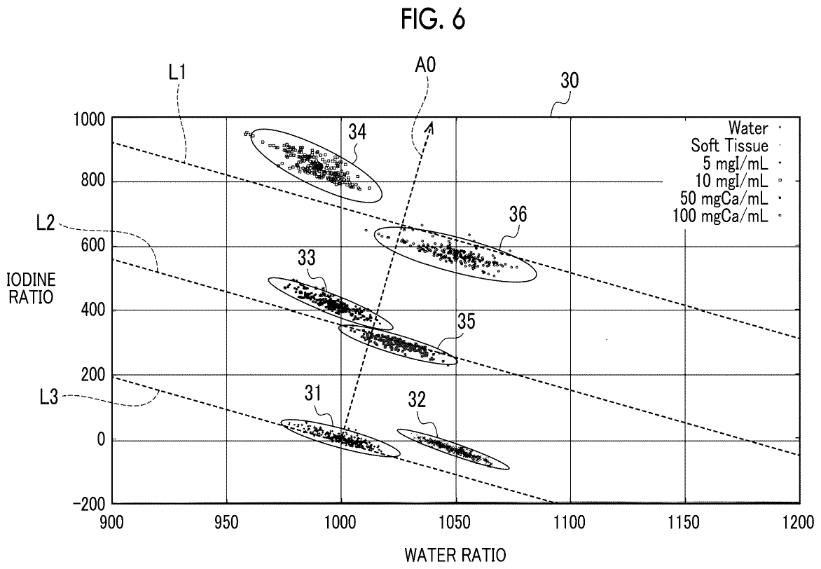

FIG. 6 is a scatter diagram to which the contour lines are added. In FIG. 6, three contour lines L1 to L3 are shown by a broken line. As shown in FIG. 6, the contour lines L1 to L3 are parallel to the longitudinal direction of the elliptical regions 31 to 36. In FIG. 6, an arrow A0 orthogonal to the contour lines L1 to L3 indicates a direction in which the pixel values of the virtual monochromatic image change.

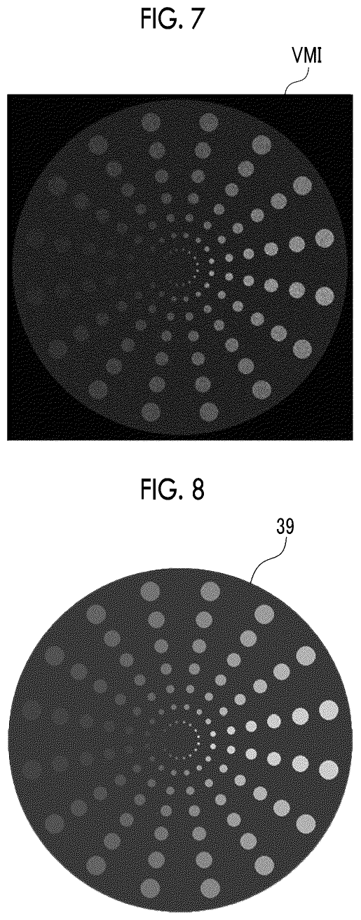

The first derivation unit 41 derives a virtual monochromatic image VMI consisting of a brightness component in which, for example, the pixel value of each pixel is a value of 0 to 255 by applying a predetermined window width WW and a window level WL to the derived virtual monochromatic image. FIG. 7 is a diagram showing the virtual monochromatic image consisting of the brightness component. In the description of FIG. 7 and subsequent figures, the description will be made using an image acquired by imaging a phantom described below.

In the virtual monochromatic image VMI, the difference in density of the substance can be represented by the brightness. However, in a direction orthogonal to the direction in which the pixel value of the virtual monochromatic image VMI changes in the scatter diagram 30, that is, in a direction of the contour line of the pixel value, the brightness (that is, the density) is the same even in a case in which the types of the substances are different, so that it is difficult to distinguish the types of the substances.

In the virtual monochromatic image VMI, the difference in the substance can be represented by the brightness. The brightness component representing the difference in substance in the virtual monochromatic image VMI is an example of the first information of the present disclosure, and the virtual monochromatic image is an example of the first image of the present disclosure. However, in a direction orthogonal to the direction in which the pixel value of the virtual monochromatic image VMI changes in the scatter diagram 30, that is, in a direction of the contour line of the pixel value, the brightness is the same even in a case in which the substances are different, so that it is difficult to distinguish the substances.

The second derivation unit 42 derives a substance type index image representing the type of the substance included in the virtual monochromatic image VMI by using the substance decomposition information derived based on the distribution of the signal values in the direction intersecting the direction in which the pixel value of the virtual monochromatic image VMI changes in the scatter diagram 30. In the present embodiment, the direction intersecting the direction in which the pixel value of the virtual monochromatic image VMI changes is a direction orthogonal to the direction in which the pixel value changes. The substance decomposition information is derived in advance by calibration.

A phantom is used to derive the substance classification information. The phantom is created by inserting a material imitating water and iodine, which are reference substances, into a cylindrical material (for example, acrylic) imitating soft tissue of the human body in different concentrations and different sizes in a major axis direction of the cylinder. FIG. 8 is a diagram showing a structure of the phantom. FIG. 8 is a cross-sectional view of a phantom 39 in a cross section perpendicular to a major axis of the cylinder. The upper half of the phantom 39 shown in FIG. 8 is made of a material imitating iodine, and the lower half is made of a material imitating water. The material imitating the reference substance used in the phantom 39 is not limited to water and iodine, and a material imitating a bone (for example, calcium) as the reference substance may be used.

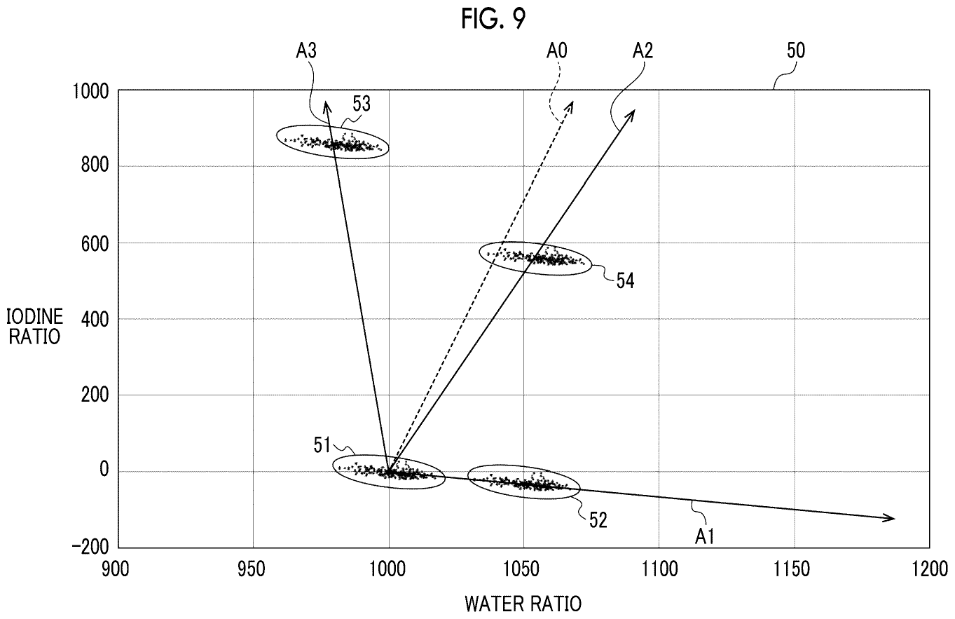

In the derivation of the substance decomposition information, the phantom 39 is imaged by the CT apparatus 2 to derive the base substance image of water and the base substance image of iodine in the same manner as described above, and the scatter diagram is derived by using the base substance image of water and the base substance image of iodine. Then, the substance decomposition information is derived by using the derived scatter diagram. FIG. 9 is a diagram for describing the derivation of the substance decomposition information. The scatter diagram shown in FIG. 9 is derived by imaging a phantom using a material imitating calcium in addition to water and iodine for the sake of description. In a scatter diagram 50 shown in FIG. 9, the signal values of water, soft tissue, iodine, and calcium for the substance constituting the phantom are distributed in regions 51 to 54. The region 51 is a distribution of the signal value of water, the region 52 is a distribution of the signal value of the soft tissue, the region 53 is a distribution of the signal value of iodine, and the region 54 is a distribution of the signal value of calcium.

As shown in FIG. 9, in the derived scatter diagram, a water ratio of 1000 and an iodine ratio of 0 that are substantially at the center of the region 51 in which the signal value of water is distributed are set as an origin, and a first reference axis A1 extending from the origin in a direction of the region 52 in which the signal value of the soft tissue is distributed, a second reference axis A2 extending from the origin in a direction of the region 54 in which the signal value of calcium is distributed, and a third reference axis A3 extending from the origin in a direction of the region 53 in which the signal value of iodine is distributed are derived as the substance classification information. In FIG. 9, the broken line arrow A0 indicates the direction in which the pixel values of the virtual monochromatic image VMI change.

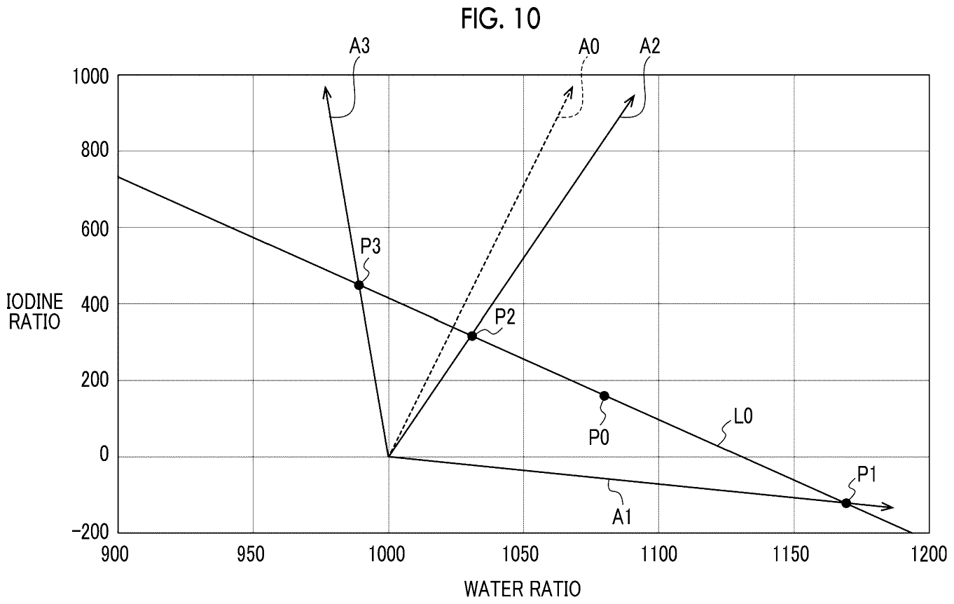

The second derivation unit 42 derives the substance type index image, which is the substance type image, by using the first to third reference axes A1 to A3 derived as shown in FIG. 9, that is, the substance classification information. The type of the substance is an example of the second information of the present disclosure, and the substance type index color image, which is the substance type image, is an example of the second image of the present disclosure. FIG. 10 is a diagram for describing the derivation of the substance type index image. As shown in FIG. 10, it is assumed that a pixel value of a certain pixel in the virtual monochromatic image VMI is P0. The pixel value P0 can be calculated from the values of water and iodine. The second derivation unit 42 sets a contour line L0 of the VMI signal value passing through the pixel value P0 in the scatter diagram. The contour line L0 extends in a direction orthogonal to the arrow A0, which is the direction in which the pixel values of the virtual monochromatic image VMI change. The second derivation unit 42 derives intersections P1 to P3 between the contour line L0 and the first reference axis A1, the second reference axis A2, and the third reference axis A3.

The second derivation unit 42 assigns an index representing the type of the substance to the derived intersections P1 to P3. For example, an index of “1” is assigned to the intersection P1, an index of “2” is assigned to the intersection P2, and an index of “3” is assigned to the intersection P3. The index of “1” represents the soft tissue, the index of “2” represents the calcium, and the index of “3” represents the iodine. In addition, the second derivation unit 42 assigns a value of 1 to 2 as the index in a range between the intersection P1 and the intersection P2 according to a distance from the intersection P1 and the intersection P2, and assigns a value of 2 to 3 as the index in a range between the intersection P2 and the intersection P3 according to a distance from the intersection P2 and the intersection P3. An index of “1” is assigned to the contour line L0 on the right side from the intersection P1, and an index of “3” is assigned to the contour line L0 on the left side from the intersection P3. As a result, for example, 1.7 is assigned as the index for the pixel value P0 shown in FIG. 10.

As a result, information having a meaning of the substance type can be assigned in a direction orthogonal to the direction in which the pixel values of the virtual monochromatic image VMI change in the scatter diagram (a direction in which the VMI signal value does not change).

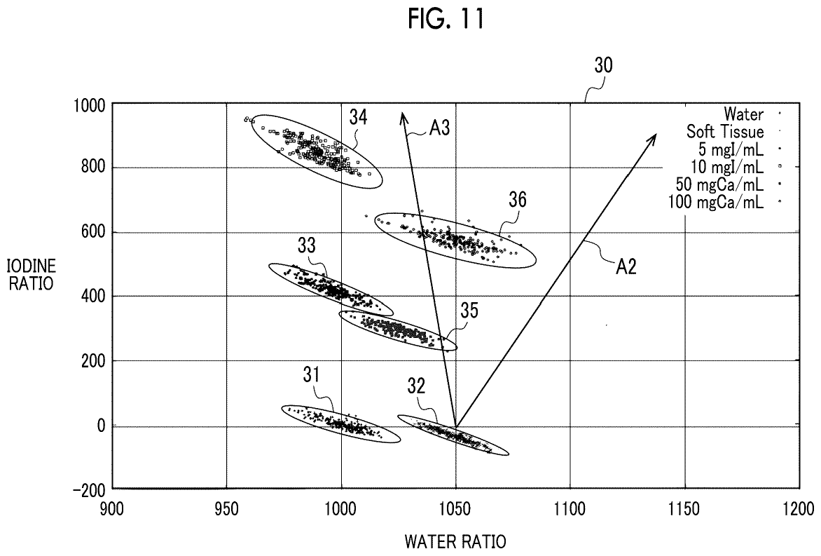

The second derivation unit 42 assigns the index representing the type of the substance to all pixels of the virtual monochromatic image VMI by using the substance decomposition information shown in FIG. 10. Then, the substance type index image is derived based on the assigned index. In a case in which the subject His imaged, the signal value of the soft tissue may be used as the origin in the scatter diagram. In this case, as an example, as shown in FIG. 11, the substance type index image may be derived by assigning the second reference axis A2 and the third reference axis A3, which are obtained by the second derivation unit 42, to the origin without changing the inclination.

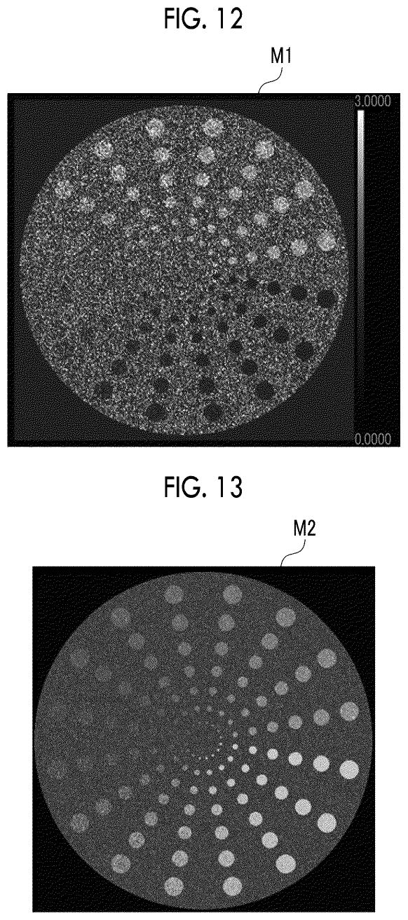

FIG. 12 is a diagram showing the substance type index image. In a case in which the substance type index image M1 shown in FIG. 12 is viewed alone, a value of 0 to 255 may be assigned to the index of 1 to 3 assigned to each pixel. As shown in FIG. 12, in the substance type index image M1, the type of the substance can be distinguished and recognized by the concentration. On the other hand, the substance type index image M1 has a lot of noise.

The third derivation unit 43 derives a substance type index color image M2 by assigning a hue different depending on the type of the substance to the substance type index image M1. FIG. 13 is a diagram showing the substance type index color image. In FIG. 13, red is assigned to iodine, and green is assigned to water. As shown in FIG. 13, in the substance type index color image M2, the type of the substance can be recognized by the concentration and the color. However, the human eye has a characteristic that the brightness felt according to the type of the color is different. The perceived brightness is often called brightness (luminance), depending on the field.

For example, it is said that the red primary color (R, G, B)=(255, 0, 0) has a brightness of about 0.3 times the white (R, G, B)=(255, 255, 255). The brightness can be represented by, for example, brightness=0.3× R+0.6× G+0.1×B using RGB. Therefore, in a method of simply providing the primary color (maximum chroma saturation color) depending on the hue, the brightness is not maintained, and the perceived brightness varies depending on the type of the color.

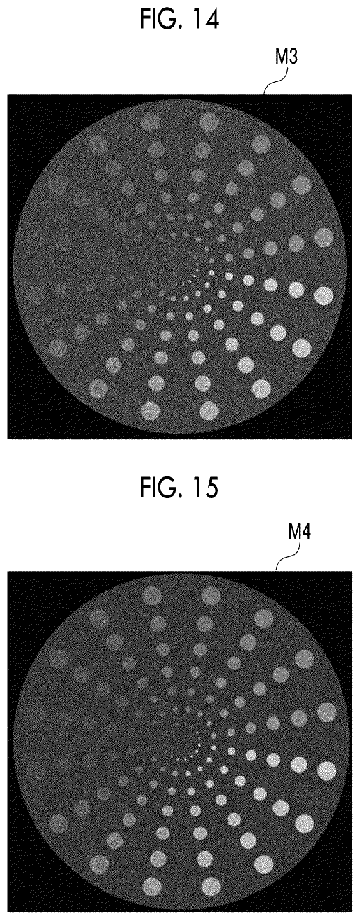

In order to solve this problem, in the present embodiment, a certain brightness is set as a unified brightness, and the color is replaced with a corresponding color (uniform brightness color) having the brightness for each hue. In a case in which the color is assigned based on the equation for obtaining the brightness from the above-described RGB, the maximum brightness (255) cannot be maintained. Therefore, the color is assigned based on any brightness value of 1 times or less with respect to the maximum brightness. The reference brightness value is set as the uniform brightness value. The lower the multiplier of the uniform brightness value, the more vivid, that is, the higher the saturation color can be assigned, but a trade-off occurs in which the overall brightness is reduced. As a value having a good balance between the chroma saturation and the brightness, for example, 0.8 times (brightness 204) can be used. In a case in which red is provided as the hue, the uniform brightness color (255, 182, 182) may be provided from the primary color (255, 0, 0), in a case in which green is provided as the hue, the uniform brightness color (126, 255, 126) may be provided from the primary color (0, 255, 0), and in a case in which blue is provided as the hue, the uniform brightness color (198, 198, 255) may be provided from the primary color (0, 0, 255). As a result, the brightness can be maintained at 0.8 times (brightness 204), and the difference in brightness due to the hue can be eliminated. It is also possible to use a brightness-unified color corresponding to any hue other than the three primary colors of RGB. The fourth derivation unit 44 derives a brightness reflecting substance color image M3 in which the brightness of the virtual monochromatic image VMI is reflected, for the substance type index color image M2. The brightness reflecting substance color image M3 is an example of the information image of the present disclosure. FIG. 14 is a diagram showing the brightness reflecting substance color image. As shown in FIG. 14, the brightness reflecting substance color image M3 has an effect of avoiding the brightness change according to the color by performing the setting of the color while maintaining the brightness of the virtual monochromatic image VMI, and not degrading the density information represented by the brightness of the virtual monochromatic image VMI.

On the other hand, in a low concentration (VMI) range, the image has a lot of noise in which red and green randomly appear for each pixel.

The fifth derivation unit 45 derives a chroma saturation reduction brightness reflecting substance color image M4 by reducing the chroma saturation as the signal value of the virtual monochromatic image VMI decreases for the brightness reflecting substance color image M3. In order to do this, the fifth derivation unit 45 derives reliability information in which the value decreases as the pixel value of each pixel of the virtual monochromatic image VMI decreases. The reliability information is derived by assigning a value of 1 to a pixel having a pixel value equal to or larger than a predetermined threshold value Th1, assigning a value of 0 to a pixel having a pixel value equal to or smaller than a predetermined threshold value Th2 (Th1>Th2), and assigning a value of 0 to 1 to a pixel having a signal value of the virtual monochromatic image VMI larger than Th2 and smaller than Th1 in accordance with the magnitude of the pixel value. The chroma saturation-decreased brightness reflecting substance color image M4 is also an example of the information image of the present disclosure.

The fifth derivation unit 45 reduces the chroma saturation of the brightness reflecting substance color image M3 derived by the fourth derivation unit 44 based on the reliability information. Specifically, the chroma saturation of the substance type index color image M2 is decreased as the reliability is lower (closer to 0). Here, in the virtual monochromatic image VMI, the noise is dominant as the signal value decreases. Therefore, the reliability information is an indicator representing the noise amount. Therefore, the chroma saturation-decreased brightness reflecting substance color image M4 in which the chroma saturation in the low concentration range of the substance type index color image M2 is further decreased is derived by decreasing the chroma saturation of the brightness reflecting substance color image M3 based on the reliability information. FIG. 15 is a diagram showing the chroma saturation-decreased brightness reflecting substance color image M4. As shown in FIG. 15, in the chroma saturation reduction brightness reflecting substance color image M4, the chroma saturation is reduced in the low concentration range of the brightness reflecting substance color image M3, but the noise is not noticeable.

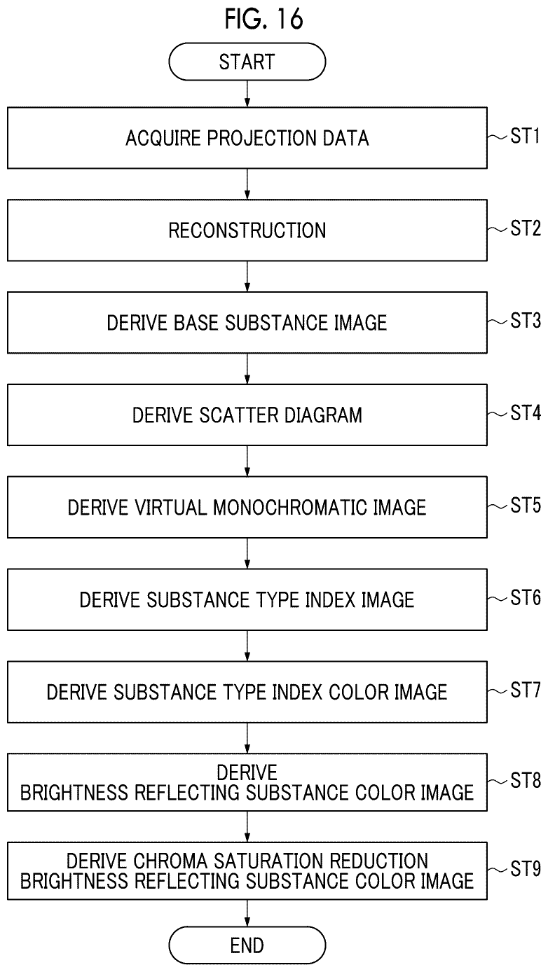

Next, processing performed in the present embodiment will be described. FIG. 16 is a flowchart showing the processing performed in the present embodiment. The data acquisition unit 21 acquires the projection data for each energy bin from the CT apparatus 2 (step ST1), and the reconstruction unit 22 derives the linear attenuation coefficient image for each energy bin by reconstructing the projection data (step ST2).

The base substance decomposition unit 23 derives the base substance images for water and iodine by using the linear attenuation coefficient corresponding to the base substance and the linear attenuation coefficient image derived by the reconstruction unit 22 (step ST3). The scatter diagram derivation unit 24 derives the scatter diagram from the base substance images for water and iodine (step ST4).

Subsequently, the first derivation unit 41 of the image derivation unit 25 derives the virtual monochromatic image VMI at an energy level at which the contour lines of the pixel values are parallel to the longitudinal direction of the distribution of the signal values of water and iodine in the scatter diagram (step ST5). The second derivation unit 42 derives the substance type index image M1 representing the type of the substance included in the virtual monochromatic image VMI by using the substance decomposition information (step ST6). The third derivation unit 43 derives a substance type index color image M2 by assigning the hue different depending on the type of the substance to the substance type index image M1 (step ST7). The fourth derivation unit 44 derives the brightness reflecting substance color image M3 in which the brightness of the virtual monochromatic image VMI is reflected, for the substance type index color image M2 (step ST8). Then, the fifth derivation unit 45 derives the chroma saturation reduction brightness reflecting substance color image M4 by reducing the chroma saturation as the signal value of the virtual monochromatic image VMI decreases for the brightness reflecting substance color image M3 (step ST9), and the processing ends.

As described above, in the present embodiment, the first image representing the first information and the second image representing the second information are acquired for the same subject, and the information image including the brightness component of the first image and the color component of the second image is derived. Specifically, the virtual monochromatic image VMI representing the difference in substance by the brightness and the substance type index image M1 representing the substance type are acquired, and the brightness reflecting substance color image M3 in which the brightness component of the virtual monochromatic image VMI is reflected in the substance type index color image M2 in which the substance type index image M1 is colored according to the substance, and the saturation-decreased brightness reflecting substance color image M4 are derived. Therefore, the type of the substance can be visible while maintaining the attenuation (density) information with only one substance type image without preparing a large number of virtual monochromatic images VMI.

In addition, since the substance type image is not based on the distribution of the signal values in the direction in which the pixel values of the virtual monochromatic image VMI change, the substance type image does not have information overlapping the virtual monochromatic image VMI. Therefore, by reflecting the brightness of the virtual monochromatic image VMI in the substance type image, the density information representing the attenuation of the substance can be recognized in addition to the types of the plurality of substances.

In addition, by deriving the substance type image having the hue different depending on the type of the substance, the types of the plurality of substances can be more easily visible.

In addition, by deriving the substance type image in which the saturation is reduced as the pixel value of the virtual monochromatic image VMI decreases, the noise in the low concentration range can be made less noticeable.

In particular, the reliability information in which the value decreases as the pixel value of the virtual monochromatic image VMI decreases is derived, and the chroma saturation is reduced based on the reliability information, so that the statistical reliability of the virtual monochromatic image VMI can be reflected in the substance type image.

In the above-described embodiment, water and iodine are used as the base substances, but the present disclosure is not limited to this, and any substance can be used as the base substance.

In addition, in the above-described embodiment, the PCCT device is used as the CT apparatus 2, but the present disclosure is not limited to this. It is needless to say that a DECT device may be used.

In the present embodiment, each process of the information processing apparatus 10 is executed by any computer. In addition, any computer may execute these processes by means of a processor as hardware, a program as software, or a combination thereof. In that case, the processor is configured to execute various types of processing in the information processing apparatus 10 of the present embodiment in cooperation with the program and can function as each unit or each means in the present embodiment. Additionally, the execution order of the process by the processor is not limited to the order described above and may be changed as appropriate. Any computer may be a general-purpose computer, a computer for a specific application, a workstation, or another system capable of executing each process.

The processor may be configured using one or more pieces of hardware, and the type of hardware is not limited. For example, the processor may be configured using hardware, such as a central processing unit (CPU), a micro processing unit (MPU), a programmable logic device, such as a field programmable gate array (FPGA), a dedicated circuit that is used to execute specific processing, such as an application-specific integrated circuit (ASIC), a graphic processing unit (GPU), or a neural processing unit (NPU). In addition, the type of hardware may be a combination of different types of hardware. In a case where a plurality of pieces of hardware are configured to execute one or more processes of a certain processor, the plurality of pieces of hardware may be present in devices physically separated from each other or may be present in the same device. Additionally, in any of the embodiments, the order of each process by the processor is not limited to the order described above and may be changed as appropriate. The hardware is configured using an electrical circuit (circuitry) in which circuit elements, such as semiconductor elements, are combined, or the like.

Further, the program may be software, such as firmware or a microcode. In addition, the program may be, for example, a program module group, and each function thereof may be implemented by a processor configured to execute the corresponding function. The program may be a program code or a plurality of code segments stored in one or more non-transitory computer-readable media (for example, storage media, other storages, or the like). The program may be distributed and stored across a plurality of non-transitory computer-readable media that are present in devices physically separated from each other. The program code or code segments may represent any combination of procedures, functions, subprograms, routines, subroutines, modules, software packages, classes, or commands, data structures, or program statements. The program code or the code segment may be connected to another code segment or a hardware circuit by transmitting and receiving information, data, an argument, a parameter, or contents of a memory.

Additionally, in the above-described embodiment, an aspect has been described in which the information processing program 12 is stored (installed) in advance in the storage 13, but the present disclosure is not limited to this aspect. The information processing program 12 may be provided in a form recorded on a recording medium, such as a compact disc read-only memory (CD-ROM), a digital versatile disc read-only memory (DVD-ROM), and a universal serial bus (USB) memory. Alternatively, the information processing program 12 may be provided in a form that can be downloaded from an external device via a network.

The technology of the present disclosure extends to all kinds of program products. The program product includes all forms of products for providing a program. For example, the program product includes a program provided through a network such as the Internet, a non-transitory computer-readable recording medium, such as a CD-ROM, a DVD, and a USB memory in which the program is stored, and the like.

Hereinafter, the supplementary claims of the present disclosure will be described.

### (Supplementary Claim 1)

An information processing apparatus comprising:

a processor, wherein the processor is configured to:

acquire a first image representing first information and a second image representing second information for the same subject; and

derive an information image including the first information as a brightness component and including the second information as a color component.

### (Supplementary Claim 2)

The information processing apparatus according to supplementary claim 1, wherein the first image is a virtual monochromatic image derived at an energy level at which contour lines of pixel values are parallel to a longitudinal direction of a distribution of signal values of a first base substance and a second base substance in a scatter diagram in which the first base substance and the second base substance serve as axes.

### (Supplementary Claim 3)

The information processing apparatus according to supplementary claim 2, wherein the second image is a substance type image representing a type of a substance in the virtual monochromatic image, which is derived by using substance decomposition information derived based on a distribution of the signal values in a direction intersecting a direction in which the pixel values of the virtual monochromatic image change in the scatter diagram.

### (Supplementary Claim 4)

The information processing apparatus according to supplementary claim 3, wherein the intersecting direction is an orthogonal direction.

### (Supplementary Claim 5)

The information processing apparatus according to supplementary claim 4, wherein the first information is a brightness component of the virtual monochromatic image, and the second information is information representing the type of the substance in the substance type image.

### (Supplementary Claim 6)

An information processing method comprising:

via a computer,
acquiring a first image representing first information and a second image representing second information for the same subject; and
deriving an information image including the first information as a brightness component and including the second information as a color component.

### (Supplementary Claim 7)

An information processing program causing a computer to execute:
a procedure of acquiring a first image representing first information and a second image representing second information for the same subject; and
a procedure of deriving an information image including the first information as a brightness component and including the second information as a color component.

What is claimed is:

1. An information processing apparatus comprising:
a processor,
wherein the processor is configured to:
acquire a first image representing first information and a second image representing second information for the same subject; and
derive an information image including the first information as a brightness component and including the second information as a color component.

2. The information processing apparatus according to claim 1,
wherein the first image is a virtual monochromatic image derived at an energy level at which contour lines of pixel values are parallel to a longitudinal direction of a distribution of signal values of a first base substance and a second base substance in a scatter diagram in which the first base substance and the second base substance serve as axes.

3. The information processing apparatus according to claim 2,
wherein the second image is a substance type image representing a type of a substance in the virtual monochromatic image, which is derived by using substance decomposition information derived based on a distribution of the signal values in a direction intersecting a direction in which the pixel values of the virtual monochromatic image change in the scatter diagram.

4. The information processing apparatus according to claim 3,
wherein the intersecting direction is an orthogonal direction.

5. The information processing apparatus according to claim 4,
wherein the first information is a brightness component of the virtual monochromatic image, and the second information is information representing the type of the substance in the substance type image.

6. An information processing method comprising:
via a computer,
acquiring a first image representing first information and a second image representing second information for the same subject; and
deriving an information image including the first information as a brightness component and including the second information as a color component.

7. A non-transitory computer-readable storage medium that stores an information processing program causing a computer to execute:
a procedure of acquiring a first image representing first information and a second image representing second information for the same subject; and
a procedure of deriving an information image including the first information as a brightness component and including the second information as a color component.
